(19) United States
(12) Patent Application Publication
(10) Pub. No.: US 2012/0009612 A1
(43) Pub. Date: Jan. 12, 2012

(54) METHOD TO DETECT AND MONITOR COMPARTMENT SYNDROME

(76) Inventor: Thomas J. Sitzman, Middleton, WI (US)

(21) Appl. No.: 12/984,091

(22) Filed: Jan. 4, 2011

Related U.S. Application Data
(63) Continuation-in-part of application No. 12/773,389, filed on May 4, 2010.
(60) Provisional application No. 61/175,314, filed on May 4, 2009.

(51) Int. Cl. C12Q 1/54

(52) U.S. Cl. 435/14

(57) ABSTRACT

Disclosed is a method of detecting and/or monitoring ischemia in a muscle tissue or compartment. The method includes the steps of measuring in real time or near-real time interstitial glucose concentration, or rate of change of interstitial glucose concentration over time, or both, in the muscle tissue or compartment. A reduced glucose concentration or a negative rate of change of glucose concentration in the tissue or compartment as compared to a control glucose concentration or rate of change indicates ischemia in the muscle or compartment.

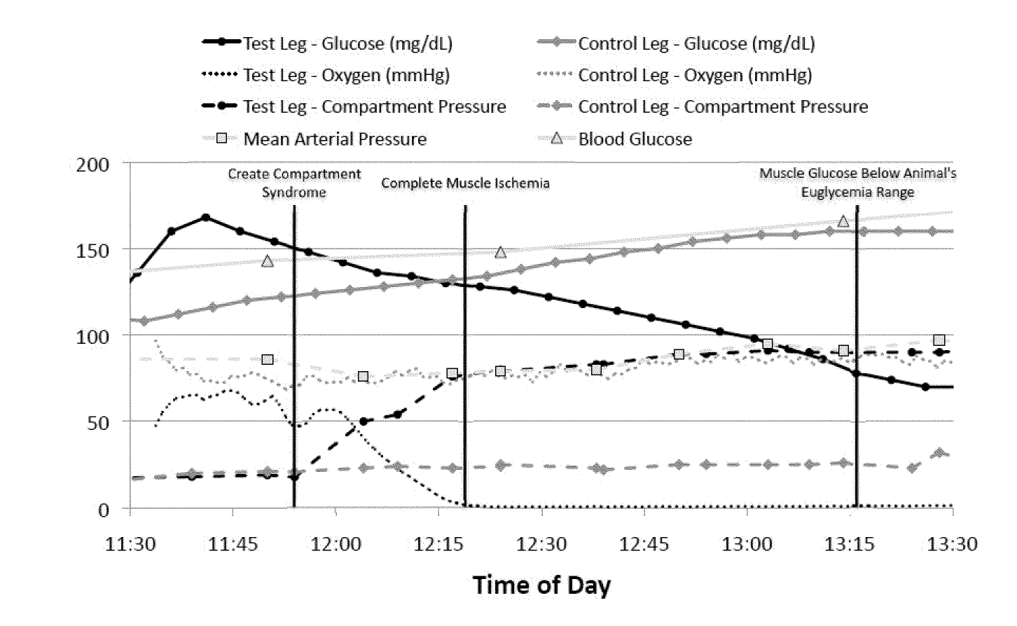

### CROSS-REFERENCE TO RELATED APPLICATIONS

This is a continuation-in-part of co-pending application Ser. No. 12/773,389, filed May 4, 2010, which claims priority to provisional application Ser. No. 61/175,314, filed May 4, 2009, both of which are incorporated herein by reference.

### BACKGROUND

A free tissue transfer (or a “free flap” transfer) is a procedure wherein an isolated and specific region of the body (for example, skin, fat, muscle, or bone), and its associated vasculature, is excised from one region of the body and transferred to another region of the body. The excised tissue is then reattached and the arterial and venous vessels reattached to establish circulation in the transferred tissue. This ability to transplant living tissue from one region of the body to another has greatly facilitated the reconstruction of complex defects. As used herein, the terms “free flap” and “free tissue transfer” are synonymous. Both terms are used to describe the movement of tissue from one site on the body to another. The word “free” indicates that the tissue, along with its blood supply, is detached from the original location (the donor site) and then transferred to another location (the recipient site).

Free tissue transfer has become commonplace in many centers around the world. The numerous advantages include stable wound coverage, improved aesthetic and functional outcomes, and minimal donor site morbidity. Since the introduction of free tissue transfer in the 1960s, the success rate has improved substantially

Since the time free tissue transfers were first developed, it has become possible to surgically repair increasingly larger and more complex tissue defects. For example, breast reconstruction after a mastectomy is now an essentially routine procedure. However, despite myriad advances in surgical techniques and instrumentation, ischemia and the subsequent necrosis of the transferred tissue remains problematic in a small, but significant percentage of patients. The same is true when transplanting other tissues and organs. It has been more than 50 years since the first successful kidney transplant (in 1954), yet ischemia in transplanted tissue continues to be a root cause of morbidity and mortality in a significant number of patients.

As used herein, the term “ischemia” means a restriction in blood supply due to any cause. In the context of a free tissue transfer, ischemia generally takes the form of clotting in the blood vessels after the flap is excised from the donor site and prior to, or just after, the flap is transplanted at the recipient site. Ischemia damages the tissue, with the ultimate result being tissue necrosis. Ischemia thus requires that the transplanted tissue be removed.

Conventionally, in the free tissue transfer of skin, the patient is monitored post-surgery to detect ischemia in the transplanted tissue. This typically involves a gross inspection of the transplant for color, temperature, and pulse, as well as stethescopic or ultrasonic auscultation to detect arterial and venous blood flow. The primary drawback of simply monitoring the patient in this fashion is that by the time any ischemia is detected (typically within 72 hours after surgery), it is often too late to do anything about it. At that point, the only course of action is to remove the transplanted tissue.

Compartment syndrome is a painful condition that occurs when pressure within a muscle or other compartment builds to dangerous levels. The increased pressure decreases blood flow to the effected area, which prevents nourishment and oxygen from reaching nerve and muscle cells. In short, compartment syndrome occurs when muscle swelling from an injury cuts off blood flow to a region of the body, usually an extremity. The resulting tissue ischemia can lead to muscle death, which can lead to a severely compromised limb

Compartment syndrome can be either acute or chronic. Acute compartment syndrome is a medical emergency. It is usually caused by a severe traumatic injury, such as a car or motorcycle accident, a crush injury, or a broken bone. Rarely, it develops after a relatively minor injury. For example, tibia fractures are the most common cause of acute compartment syndrome within the muscles of the calf. Without treatment, it can lead to permanent muscle damage. It can also occur in other compartments in the leg, as well as in the arms, hands, feet, and buttocks. To relieve the intra-compartmental pressure, a fasciotomy is performed: the skin and the fascia covering the affected compartment are incised to relieve the pressure. If the swelling is severe, the incision is left open—a morbid procedure with high rates of complications. The incision is surgically repaired when swelling subsides. If the edges of the incision cannot be brought together after the swelling has subsided, a skin graft is used to close the wound.

Chronic compartment syndrome, also known as exertional compartment syndrome, is not normally a medical emergency. It is most often caused by aggressive athletic exertion. The pain and swelling of chronic compartment syndrome is caused by exercise. Athletes who participate in activities with repetitive motions, such as running, biking, or swimming, are more likely to develop chronic compartment syndrome. This is usually relieved by discontinuing the exercise, and is usually not dangerous.

In both acute and chronic compartment syndrome, prolonged tissue ischemia is the root cause of potentially permanent damage to the muscle tissues within the affected compartment.

There are two instrumental approaches that have been used to monitor tissue ischemia. The first approach uses blood oximetry; the second approach uses micro-dialysis. Both approaches, however, suffer from drawbacks in terms of cost, sensitivity, and real-time functionality.

ViOptix, Inc., of Fremont, Calif., markets a proprietary tissue oximetry technology which enables non-invasive, direct, real-time measurement of local tissue oxygen saturation. Oxygen, of course, is a key parameter in many clinical areas such as tissue viability, revascularization, cancer management, circulatory exploration and muscle assessment. The level of oxygen saturation can thus be used to measure tissue ischemia. See, for example, U.S. Pat. No. 7,247,142, issued Jul. 24, 2007, and U.S. Pat. No. 7,525,647, issued Dec. 21, 2007, both of which are assigned to ViOptix, Inc.

Micro-dialysis is a technique used to determine the chemical components of the fluid in the extracellular space of tissues. A microdialysis probe, which is inserted into the tissue, is a tiny tube made of a semi-permeable membrane. A dialysate solution is pumped through the dialysis probe, and chemical entities in the extracellular space diffuse into the dialysate. The dialysate is then collected and analyzed to determine the identities and concentrations of molecules that were in the extracellular fluid. In the context of free tissue transfers, it has been found that simultaneously monitoring the level of glucose, lactate, pyruvate and glycerol correlates well with the presence of ischemia in the free tissue. See, for example, Rojdmark et al. (January 1992) “Comparing metabolism during ischemia and reperfusion in free flaps of different tissue composition,” European Journal of Plastic Surgery 24(7): 349-355. In this study, the interstitial kinetics of glucose, lactate, pyruvate and glycerol were studied during ischemia and reperfusion in human free flaps of different tissue compositions (skin, muscle and adipose tissue). The concentrations of the substances were determined repeatedly during ischemia and reperfusion, and laser doppler flowmetry was used to document revascularization. Microdialysis catheters were placed in the flap tissue and in similar, non-operated control tissue. The drawback of micro-dialysis, however is that it is not a real-time protocol. Like conventional visual monitoring, it can detect ischemia, but not soon enough to do anything about it.

Thus there remains a long-felt and unmet need for a sensitive, accurate, and real-time method to detect and to monitor compartment syndrome.

### BRIEF DESCRIPTION OF THE DRAWING

The sole drawing FIGURE is a graph depicting interstitial glucose levels (mg/dL) and oxygen levels (tissue oximetry) vs. time (in minutes) for compartment syndrome induced in the hind limbs of dogs and untreated control animals. Legend: (——) test leg interstitial glucose; ( . . . . ) test leg oxygen; (--) test leg compartment pressure; (—♦—) control leg interstitial glucose; ( . . . . ) control leg oxygen; (-♦-) control leg compartment pressure; (-▪-) mean arterial pressure; (-▴-) blood glucose.

### DETAILED DESCRIPTION OF THE INVENTION

Ischemia occurs when the blood supply to tissues is blocked and can be caused by a variety of trauma or disease. The lack of blood supply prevents oxygen from getting to cells and prevents waste products from being removed out of the tissue. In plastic surgery, blood vessel occlusion after free tissue transfer leads directly to transfer ischemia. In compartment syndrome, once the intra-compartment pressure equals or exceeds the mean arterial pressure of the affected subject, blood flow to the compartment ceases and ischemia begins. This ischemia must be corrected within roughly six hours of onset to prevent either complete tissue flap loss, or irreversible muscle damage. If detected early enough, vessels can be unblocked, and the tissue transfer can be saved. In the case of compartment syndrome, intra-compartmental pressure can be lowered via a fasciotomy or other surgical intervention to lower the intra-compartmental pressure. However, for the ischemia to be addressed, it is imperative to detect where and when the blockage is occurring as quickly as possible in order to target treatment. The same situation applies in other transplant procedures. For example, early detection of thrombosis after kidney transplant can salvage the transplanted organ if blood flow is re-established quickly. Again, the ischemia must be detected quickly to have any probability of salvaging the transplant or avoiding permanent muscle damage. Similarly, monitoring ischemia in the brain after stroke or other neurological trauma can affect the strategy selected regarding re-perfusion of the damaged area. (Repeat cycles of ischemia and re-perfusion can cause further damage to the brain of a stroke patient and should be avoided.) Re-perfusion needs to be tightly controlled in these patients. That can only be done if ischemia can be monitored quickly, precisely, and accurately, in real time or near real time.

The present invention is a method of monitoring absolute glucose levels and the rate of change in glucose levels in tissue in general and muscle tissue in particular. It has been found that both the absolute glucose level and the rate of change of glucose level in muscle tissue correlates very closely with the onset and progression of ischemia in the tissue. Using a composite log regression of both variables enables ischemia to be detected with a speed, accuracy, and precision previously unattainable. The method can be implemented using absolute glucose level as the metric, or rate of change of glucose level as the metric, or both glucose level and rate of change as the metric (preferred).

Logistical regression (i.e., log regression) analysis is well-known and will not be described in great detail herein. Very briefly, a log regression analysis uses the following logistic function: ƒ(z)=1/1+e^{-z}. The logistic function can take as a numerical input any value from negative infinity to positive infinity. Because of the logarithmic nature of the function ƒ(z), the output is confined to values between 0 and 1. The variable z represents the exposure to a pre-selected set of independent variables (in this case absolute glucose level and/or rate of change of glucose level). The function ƒ(z) represents the probability of a particular outcome (ischemia, necrosis, etc.) given the set of explanatory variables. The variable z is a measure of the total contribution (i.e., the composite value) of all the independent variables used in the model.

The variable z is usually defined as z=β₀+β₁x₁+β₂x₂+β₃x₃+ . . . β_{y}x_{y}, wherein β₀ is called the “intercept” and β₁, β₂, β₃, etc. and so on, are called the “regression coefficients” of x₁, x₂, and x₃, respectively. The intercept is the value of z when the value of all independent variables is zero (e.g., the value of z in a transplant with no risk of ischemia). Each of the regression coefficients describes the size of the contribution of that specific risk factor. A positive regression coefficient means that that explanatory variable increases the probability of the outcome, while a negative regression coefficient means that variable decreases the probability of that outcome; a large regression coefficient means that the risk factor strongly influences the probability of that outcome; while a near-zero regression coefficient means that that risk factor has little influence on the probability of that outcome. Logistic regression is a useful way of describing the relationship between one or more independent variables (e.g., absolute glucose level, rate of change of glucose level) and a binary response variable, expressed as a probability, that has only two possible values (i.e., ischemia in the transplanted tissue/organ or no ischemia). For an exhaustive treatment, see, for example, “Statistics, 4th Edition,” David Freedman, Robert Pisani, and Roger Purves, © 2007, W. W. Norton & Company, ISBN-13: 978-0393929720.

Importantly, the method described herein accurately detects and monitors arterial and venal occlusion and compartment syndrome. Detection times are very rapid, within minutes of the onset of ischemia. This is a stark contrast to microdialysis, wherein the tissue must be monitored at specific time intervals for hours. Microdialysis also suffers from having poor specificity in determining venal occlusion. The present method measures arterial occlusion with 100% sensitivity and specificity in 20 minutes or less. The method likewise detects venous occlusion with equal success.

In the preferred version of the method, glucose level and rate of change are measured using a continuous glucose monitor, such as a “Guardian”-brand glucose monitor, which is manufactured and marketed worldwide by Medtronic, Inc., Minneapolis, Minn. The “Guardian”-brand glucose monitoring device is described in U.S. Pat. No. 6,809,653, incorporated herein by reference, and in the manufacturer's User's Guides, also incorporated herein by reference. (The manufacturer's User's Guides are included as part of corresponding provisional application Ser. No. 61/175,314, filed May 4, 2009.) Several other suitable devices are commercially available, such as DexCom's “Seven Plus”-brand glucose monitor (DexCom, Inc., San Diego, Calif.), Medtronics' “Paradigm”®-brand real-time glucose monitoring system (Medtronic Diabetes, Northridge, Calif.), and Abbott's “FreeStyle Navigator”®-brand glucose monitoring system (Abbott Diabetes Care, Inc., Alameda, Calif.). See also U.S. Pat. Nos. 5,390,671; 5,391,250; 5,568,806; 5,586,553; 5,777,060; 5,779,665; 5,786,439; 5,851,197; 5,882,494; 5,954,643; 6,093,172; 6,293,925; 6,462,162; 6,520,326; 6,607,509, 7,693,560; 7,657,297; 7,654,956; 7,651,596; 7,640,048; 7,637,868; 7,632,228; 7,615,007; 7,613,491; 7,599,726; 7,591,801; 7,462,264 7,225,535; 7,670,853; 7,381,184; 7,550,069; 7,563,350; 7,582,059; 7,003,340; 7,510,564; and 7,583,990, all of which are incorporated herein by reference. Note, however, any device capable of and dimensioned and configured for measuring interstitial glucose on a continuous or semi-continuous basis may be used in the present method, whether now known or developed in the future.

The “Guardian”-brand glucose monitor includes a sensor (part nos. MMT-7002 or MMT 7003) which is a membrane-covered electrode that measures glucose levels in the interstitial space where the sensor is inserted. The sensor is operationally connected to a transmitter (part no. MMT-7703). The transmitter sends the glucose data gathered by the sensor to a monitor (part nos. CSS-7100 or CSS7100K) that displays (and stores) real-time glucose measurements, change in glucose levels, historic glucose levels, high and low glucose levels, etc. A USB-compatible, wireless radio frequency upload device (part no. MMT-7305) can also be used to download data from the transmitter directly to a programmable computer. Using this device, glucose levels in a free-flap tissue or organ to be transplanted can be transferred in real time to a computer and monitored automatically and continuously (again in real time) and an alarm sounded (automatically) if the absolute glucose level detected by the sensor dips below a preset level, or the rate of change of the glucose level exceeds a preset level.

Thus, one version of the invention is directed to a method of detecting and/or monitoring ischemia in a muscle tissue or compartment. The method comprises measuring in real time or near-real time the interstitial glucose concentration, or the rate of change of interstitial glucose concentration over time, or both, in the muscle tissue or compartment. A reduced glucose concentration or a negative rate of change of glucose concentration in the muscle tissue or compartment as compared to a control glucose concentration or rate of change indicates ischemia in the muscle tissue or compartment.

As used herein, the term “control” is used in its broadest sense to mean a “normal” or “acceptable” glucose level or a range of “normal” or “acceptable” glucose levels established on a patient-by-patient, tissue-by-tissue, and/or organ-by-organ basis. The control may also or alternatively be based on aggregate glucose values taken from a sampling of “normal patients,” “normal tissues,” and/or “normal organs” and presented in the form of a standard curve or a set of standard values that constitute acceptable glucose levels and acceptable rates of change of glucose levels. The control values are then used to establish what constitutes unacceptably low glucose levels and/or unacceptably steep rates of change in glucose levels that are indicative of ischemia in the tissue or organ tested. Control glucose values can be obtained directly from the donor patient (for an autologous free-flap transfer) or donor tissue/organ prior to transplantation (by sampling the tissue or organ to be transplanted), or from unaffected limbs in patients suspected of developing compartment syndrome in a damaged or otherwise compromised limb. Control glucose values may also be aggregated from the values taken from many different patients. The control values may be obtained in advance (e.g., by compiling a standard curve of glucose values) or contemporaneously with the treatment protocol being undertaken. In short, as used herein “control” means any protocol designed to provide a reference set of glucose level data which can be compared with data obtained from the tissue or organ that is being tested to thereby determine whether the glucose values in the tissue or organ are within an acceptable range prior to, during, and after testing or whether the levels have reached a point indicating that ischemia has taken place in the tissue or organ (and thus further medical action must be taken to relieve the ischemia before cell or tissue death occurs).

Likewise, the invention includes a method of detecting and/or monitoring ischemia in a tissue or organ comprising measuring the rate of change of glucose concentration in the tissue or organ. Here, a negative rate of change of glucose concentration in the tissue or organ (as compared to a control glucose concentration) indicates ischemia in the tissue or organ. More specifically, the method disclosed herein is particularly useful to detect and/or monitor ischemia in muscles suspected of experiencing compartment syndrome.

### EXAMPLES

The following Examples are presented to provide a more complete description of the method disclosed and claimed herein. The Examples do not limit the scope of the claimed method in any fashion.

Dogs (beagles) were used to model compartment syndrome in vivo. In each test animal, compartment syndrome was induced in one hind limb by filling the limb's muscle compartment with normal saline until the intra-compartment pressure was equal to the animal's mean arterial pressure. Maintaining the intra-compartment pressure at this level cuts of blood flow to the muscles within the compartment, thereby resulting in muscle ischemia. The other hind limb of each test animal was used for a control. The intra-compartment pressure was measured continuously during the course of each experiment, as was glucose concentration within the muscle and tissue oximetry within the compartment. Ischemia within the test limbs was confirmed via oximetry, which showed severe muscle anoxia once the intra-compartment pressure reached the animal's mean arterial pressure. In the control limbs, identical sensors were inserted (intra-compartment pressure, glucose monitor, tissue oximetry monitor), but no saline was infused into the control limbs. The results from two animals are depicted in the FIGURE.

As can be seen from the FIGURE, compartment pressure in the treated limbs (broken line with circles) rises quickly when the saline pressure equals the mean arterial pressure of the animal. At the same time, oxygen levels within the test limbs (broken line) plummets to zero approximately 20 minutes after onset of compartment syndrome (just after time point 12:15, marked with a vertical line titled “Complete Muscle Ischemia”). In the control limbs, compartment pressure (broken line with diamonds) and oxygen levels (broken line) remain essentially constant throughout the experiment.

In the control animals, glucose levels (solid line with diamonds) rose slightly throughout the course of the experiments. In contrast, in the limbs where compartment syndrome was induced, glucose levels (solid line with circles) start to fall immediately after the onset of compartment syndrome and continue to drop throughout the course of the experiment. At the onset of compartment syndrome, glucose in the test limbs drops at an essentially linear rate. See the solid line with circles in the area of the FIGURE between “Complete Muscle Ischemia” and “Muscle Glucose Below Animal's Euglycemia Range.” (Euglycemia is the normal range of glucose within an animal's blood. When the amount of glucose in a muscle drops below the animal's euglycemia range, the muscle will no longer function properly.)

Thus, as shown by the FIGURE, both the absolute amount of glucose within the muscle tissue, as well as the rate of change of glucose concentration, are both indicators of the presence (or absence) of compartment syndrome. A decreased amount of glucose or a negative rate of change of glucose concentration in the muscle tissue indicates the present of compartment syndrome in the muscle tissue tested.

What is claimed is:

1. A method of detecting and/or monitoring ischemia in a muscle tissue or compartment, the method comprising:
measuring in real time or near-real time interstitial glucose concentration, or rate of change of interstitial glucose concentration over time, or both, in the muscle tissue or compartment, wherein a reduced glucose concentration or a negative rate of change of glucose concentration in the muscle tissue or compartment as compared to a control glucose concentration or rate of change indicates ischemia in the muscle tissue or compartment.

2. The method of claim 1, comprising measuring interstitial glucose concentration.

3. The method of claim 2, wherein a measured interstitial glucose concentration of less than about 100 mg/dL indicates ischemia in the muscle tissue or compartment.

4. The method of claim 1, comprising measuring rate of change of interstitial glucose concentration over time.

5. The method of claim 4, wherein a measured rate of change of interstitial glucose concentration steeper than about −2 mg/dL/minute indicates ischemia in the muscle tissue or compartment.

6. The method of claim 4, wherein a measured rate of change of interstitial glucose concentration steeper than about −3 mg/dL/minute indicates ischemia in the muscle tissue or compartment.

7. The method of claim 4, wherein a measured rate of change of interstitial glucose concentration steeper than about −4 mg/dL/minute indicates ischemia in the muscle tissue or compartment.

8. The method of claim 1, comprising measuring interstitial glucose concentration and rate of change of interstitial glucose concentration over time.

9. The method of claim 8, wherein a measured interstitial glucose concentration of less than about 118 mg/dL and a measured rate of change of interstitial glucose concentration steeper than about −2 mg/dL/minute indicates ischemia in the muscle tissue or compartment.

10. The method of claim 9, wherein a measured rate of change of interstitial glucose concentration steeper than about −3 mg/dL/minute indicates ischemia in the muscle tissue or compartment.

11. The method of claim 9, wherein a measured rate of change of interstitial glucose concentration steeper than about −4 mg/dL/minute indicates ischemia in the muscle tissue or compartment.

12. A method of detecting and/or monitoring ischemia in a muscle suspected of being subject to compartment syndrome, the method comprising:
measuring in real time or near-real time interstitial glucose concentration, or rate of change of interstitial glucose concentration over time, or both, in the muscle, wherein a reduced glucose concentration or a negative rate of change of glucose concentration in the free tissue transfer as compared to a control glucose concentration or rate of change indicates ischemia in the muscle.

13. The method of claim 12, comprising measuring interstitial glucose concentration.

14. The method of claim 13, wherein a measured interstitial glucose concentration of less than about 118 mg/dL indicates ischemia in the muscle.

15. The method of claim 12, comprising measuring rate of change of interstitial glucose concentration over time.

16. The method of claim 15, wherein a measured rate of change of interstitial glucose concentration steeper than about −2 mg/dL/minute indicates ischemia in the muscle.

17. The method of claim 16, wherein a measured rate of change of interstitial glucose concentration steeper than about −3 mg/dL/minute indicates ischemia in the muscle.

18. The method of claim 16, wherein a measured rate of change of interstitial glucose concentration steeper than about −4 mg/dL/minute indicates ischemia in the muscle.

19. The method of claim 12, comprising measuring interstitial glucose concentration and rate of change of interstitial glucose concentration over time.

20. The method of claim 19, wherein a measured interstitial glucose concentration of less than about 118 mg/dL and a measured rate of change of interstitial glucose concentration steeper than about −2 mg/dL/minute indicates ischemia in the muscle.

21. The method of claim 20, wherein a measured rate of change of interstitial glucose concentration steeper than about −3 mg/dL/minute indicates ischemia in the muscle.

22. The method of claim 20, wherein a measured rate of change of interstitial glucose concentration steeper than about −4 mg/dL/minute indicates ischemia in the muscle.
